(19) United States
(12) Patent Application Publication
(10) Pub. No.: US 2007/0203109 A1
(43) Pub. Date: Aug. 30, 2007

(54) Compounds And Compositions For Treatment Of Cancer

(75) Inventors: Timothy H. Ward, London (GB); Malcolm Ranson, London (GB)

Correspondence Address: SWANSON & BRATSCHUN, L.L.C., 8210 SOUTHPARK TERRACE, LITTLETON, CO, 80120, US

(73) Assignee: CANCER RESEARCH TECHNOLOGY LIMITED, London Greater London (GB)

(21) Appl. No.: 11/568,633

(22) PCT Filed: May 6, 2005
(86) PCT No.: PCT/GB05/01804

§ 371 Date: Nov. 3, 2006

(30) Foreign Application Priority Data
Jan. 18, 2005 (GB) 0501012.9
May 6, 2004 (GB) 0410143.2

(51) Int. Cl. A61K 31/396

(52) U.S. Cl. 514/183

(57) ABSTRACT

The present invention relates to the use of a therapeutically effective amount of 2,5-diaziridinyl-3-(hydroxymethyl)-6-methyl-1,4-benzoquinone (RH1), in the manufacture of a medicament for the treatment of a cancerous condition.

### FIELD OF THE INVENTION

The present invention relates generally to the field of anti-tumour compounds, and particularly, although not exclusively, to the compound 2,5-diaziridinyl-3-hydroxymethyl-6-methyl-1,4-benzoquinone (RH1) and to its use in treating cancerous conditions.

### BACKGROUND TO THE INVENTION

DT-Diaphorase (DTD) was first isolated in 1958 and has been referred to by a variety of names including NAD(P)H: quinone oxidoreductase (EC 1.6.99.2)(NQO1), vitamin K reductase, phylloquinone reductase, menadione reductase and nicotinamide menaione oxidoreductase.

DTD is a flavoprotein which exists as a dimer. Both subunits are of equal size, have MW of 32000 Dalton and have 2 FAD groups.

DTD is an obligatory two electron reductase enzyme (in contrast to the one electron reductase enzymes such as cytochrome b reductase, cytochrome P450 reductase and xanthine dehydrogenase) and utilises co-factors NADH and NADPH equally well as the electron donor.

DTD performs a number of functions including Phase II detoxification, a detoxifying step that bypasses the formation of free radicals and protects tissue against mutagens, carcinogens and cytotoxics. DTD also metabolises quinones (e.g. originating from diet or the environment). In particular it can reductively activate cytotoxic antitumour quinones. Furthermore, DTD functions as a vitamin K reductase involved in hepatic post-translational modification of vitamin K. DTD is distributed throughout the body with higher levels in the liver, kidney and gastrointestinal tract.

There are four different isoforms of DTD. The best characterised is NQO1 and the gene for this isoform is located on chromosome 16. It is 274 residues long and has an ARE (antioxidant response element), AP1 site, XRE, CAT, TATA box and NFkB binding site. Binding to ARE mediates signal transduction (Faig et al. PNAS 28, 3177-82, 2000).

Elevated levels of DTD can be found in certain tumour types, compared to normal tissue (Schlager et al., Int. J. Cancer 45, 403-409, 1990). Examples of tumour types and the ratio of DTD levels are set out in table 1.

TABLE 1

| | Ratio DTD |
|---|---|
| Tumour type | (tumour:normal tissue) |
| Lung | 17.5 |
| Colon | 9.0 |
| Liver | 3.3 |
| Breast | 3.0 |
| Stomach | 0.38 |
| Kidney | 0.12 |

(Schlager et al. Int. J. Cancer 45, 403-9, 1990)

Thus, DTD is over-expressed in many cancerous tissues, in particular in non-small cell lung cancer (NSCLC).

Secondary or metastatic tissue is found to have similar DTD levels to the primary tumour.

U.S. Pat. No. 6,156,744, incorporated herein in its entirety by reference, proposes the use of 2,5-diaziridinyl-3-hydroxymethyl-6-methyl-1,4-benzoquinone (RH1) and certain esters of RH1 for the treatment of lung cancer, NSCLC, liver, breast, colon, CNS, stomach, bladder and skin cancer.

Whilst it has been suggested that certain quinones may have a role in cross-linking DNA, there is no understanding of the mechanism involved or of the structural features which may promote efficient DNA cross-linking, nor any way of predicting water solubility, toxicity and suitability as a prodrug for bioreduction.

### SUMMARY OF THE INVENTION

The present inventors have found that certain diaziridinylbenzoquinone compounds are suitable for treatment of a wide range of cancerous conditions, and that such compounds are particularly effective when administered to a patient at certain dosage levels, optionally as part of a predetermined dosage regime, and/or using certain modes of administration.

The inventors have identified compounds that not only exhibit significant DNA cross-linking ability in vivo but also low levels of toxicity and good water solubility.

Diaziridinylbenzoquinone compounds of the invention include the compound 2,5-diaziridinyl-3-hydroxymethyl-6-methyl-1,4-benzoquinone (herein called RH1) and its esters. The general chemical structure of the esters is given by Formula I and the structure of RH1 is given by Formula II:

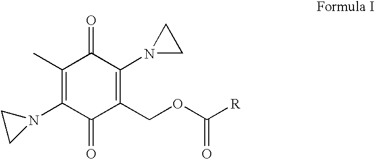

Where R can be benzoyl, acetyl, naphthoyl or protected amino acids.

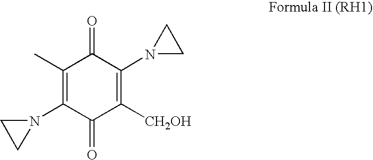

Reference herein to RH1 or compounds of Formula I includes the salts thereof, in particular pharmaceutically acceptable salts thereof.

The present inventors have found that compounds of formula I, in particular RH1, are readily activated by DTD. Although the invention is not to be limited by any particular theory, the two electron reductase activity of DTD is considered to reduce RH1 to the active hydroquinone, producing a powerful DNA cross-linking agent. This activation mechanism, in combination with the significant levels of DNA cross-linking attributable to these compounds makes them very promising anti-tumour agents.

Preferably the observed over-expression of DTD in tumours and the efficient activation of compounds of the present invention by DTD means that compound activation occurs preferentially within the tumour. As well as targeting the tumour itself, this has the advantage that the increased levels of activated quinone should not be detrimental to normal tissue, which may surround the tumour, because the activated quinones will be localised at the tumour.

Tumours with high DTD levels preferably exhibit correspondingly higher levels of DNA cross-linking when treated with compounds of the present invention, particularly RH1.

Accordingly, RH1 is a bioreductively activated drug that has been found to be an excellent substrate for DTD. DTD reduces RH1 to a hydroquinone producing a powerful cross-linking agent. RH1 is presently undergoing a Cancer Research-UK phase I trial at the Christie Hospital, Manchester, UK (PH1/089).

Compounds of the present invention, including RH1, can be thought of as pro-drugs, in the sense that they are metabolised by DTD to convert them into their active form. Thus, some aspects of the present invention relate to such prodrugs which may be activated in tumour cells by conversion to an active agent capable of treating the tumour. This may provide for selective killing of tumour cells.

Reduction of quinones by DTD may lead to production of either reactive oxygen species by autoxidation or a reactive alkylating species by rearrangement.

It has been found that compounds of the present invention, in particular RH1, can cross-link DNA. Preferably significant cross-linking (e.g. in the range 40-95%) is caused, which may lead to accumulative DNA damage. For example, compounds or compositions of the present invention, e.g. containing RH1, may be administered to cause at least 10% cross-linking in DNA, more preferably at least 20% cross-linking, even more preferably at least 30% cross-linking, still more preferably at least 40% cross-linking and most preferably at least 50% cross-linking: For example, RH1 has been found to cause up to 30% cross-linking in DNA of peripheral blood lymphocytes.

The activity of a given substance or molecule may be measured by assaying for the activity, e.g. cross-linking activity can be measured by assaying for cross-linking using the Comet-X test discussed below.

Suitably, compounds of the present invention are water soluble and have low toxicity.

It may be convenient or desirable to prepare, purify, and/or handle a corresponding salt of the compounds, preferably a pharmaceutically-acceptable salt. It is preferred that the salt is water soluble. Examples of pharmaceutically acceptable salts are discussed in Berge et al., 1977, “Pharmaceutically Acceptable Salts,” J. Pharm. Sci., Vol. 66, pp. 1-19.

Accordingly, aspects of the invention may include any known pharmaceutically acceptable salt of the compounds of the present invention.

Accordingly, compounds, compositions, uses and methods of the invention which refer to compounds of the present invention, in particular 2,5-diaziridinyl-3-hydroxymethyl-6-methyl-1,4-benzoquinone (RH1), may include salts, preferably pharmaceutically acceptable salts of the compounds, in particular of 2,5-diaziridinyl-3-hydroxymethyl-6-methyl-1,4-benzoquinone (RH1).

In a further aspect the present invention provides pharmaceutical compositions comprising diaziridinylbenzoquinone compounds of formula I or salts thereof, in particular comprising 2,5-diaziridinyl-3-hydroxymethyl-6-methyl-1,4-benzoquinone (RH1). Preferably, such compositions comprise one or more pharmaceutically acceptable carriers, adjuvants or diluents. The compounds can be formulated in any pharmaceutically acceptable formulation. Such formulations may include liquids, powders, creams, emulsions, pills, troches, suppositories, suspensions, solutions, and the like. Other excipients can also be added and are readily identified by those skilled in the art. Preferably, the compounds are soluble in aqueous solutions, are stable, and can be prepared in gram quantities. For example, formulations may be in tablet form, or suitable for injection, e.g. combined with an appropriate fluid carrier.

Medicaments and pharmaceutical compositions according to aspects of the present invention may be formulated for administration by a number of routes, including but not limited to, topical, parenteral, intravenous, intramuscular, intratumoural, intrathecal, intraocular, subcutaneous, transdermal, oral and nasal. The medicaments and compositions may be formulated in fluid or solid form, for example as an injectable composition or in tablet form. Fluid formulations may be formulated for administration by injection to a selected region of the human or animal body, typically combined with an appropriate fluid carrier.

Aspects of the present invention relate to the treatment of cancerous conditions. As such, a method of treating a cancerous condition in a patient comprising administering to said patient a therapeutically-effective amount of a compound of the present invention is provided. Thus, methods of treating patients, including human patients, having cancer are provided.

Treatment may be by administration of compounds of formula I, in particular 2,5-diaziridinyl-3-hydroxymethyl-6-methyl-1,4-benzoquinone (RH1) or compositions containing 2,5-diaziridinyl-3-hydroxymethyl-6-methyl-1,4-benzoquinone (RH1).

Suitably, the compound or pharmaceutically acceptable salt thereof is part of a composition and it is the composition that is administered.

Preferred routes of administration of the compound or composition may include one or more selected from topical, parenteral, intravenous, intramuscular, intratumoural, intrathecal, intraocular, subcutaneous, transdermal, oral and nasal. A particular formulation of the compound, e.g. RH1, may be selected to correspond to the route of administration that is to be used.

Treatment may include administration of one or more boli, and/or infusion.

Infusion, e.g. intravenous infusion, may occur over a given time period, e.g. 10-30 minutes which is sufficient to infuse the required dosage.

Dosage

Therapeutically effective amounts of the compounds can be any amount or dose sufficient to bring about the desired therapeutic effect (e.g. killing of tumour cells) and may depend, in part, on factors such as the condition, type and location of the cancerous condition being treated, as well as the size and condition of the patient. The dosages can be given as a single dose, or as several doses, for example, divided over the course of several weeks. The dosages may be administered as part of a predetermined programme of treatment.

A therapeutically effective amount may be one that produces at a given time after administration a blood, plasma or serum concentration of RH1 in the patient which is in the range 30 to 120 nM, more preferably in the range 50 to 90 nM. Still more preferably, the therapeutically effective amount may be one that produces at a given time after administration a blood, plasma or serum concentration of RH1 in the patient which is selected from one of: 30-35 nM; 35-40 nM; 40-45 nM; 45-50 nM; 50-55 nM; 55-60 nM; 60-65 nM; 65-70 nM; 70-75 nM; 75-80 nM; 80-85 nM; 85-90 nM; 90-95 nM; 95-100 nM; 100-105 nM; 105-110 nM; 110-115 nM; 115-120 nM; 120-125 nM; or 125-130 nM. The given time may be one of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59 or 60 minutes after administration or one or more of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35 or 36 hours after administration. A serum sample may comprise the fluid portion of the blood obtained after removal of the fibrin clot and blood cells.

Blood, plasma or serum concentrations may be measured immediately after infusion (i.e. at the end of infusion, t₀) or at any of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59 or 60 minutes after to or any of 1, 2, 3, 4, 5, 6, 7, 8, 9, 10, 11, 12, 13, 14, 15, 16, 17, 18, 19, 20, 21, 22, 23 or 24 hours after t₀.

A therapeutically effective amount may be one that is in the range 40 μg/m²/day to 350 μg/m²/day. Thus, preferred dosages may include one or more of at least 40 μg/m²/day; at least 80 μg/m²/day; at least 135 μg/m²/day; at least 200 μg/m²/day; at least 265 μg/m²/day; at least 350 μg/m²/day; at least 460 g/m²/day; at least 470 g/m²/day; at least 610 μg/m²/day; at least 810 μg/m²/day; at least 870 μg/m²/day; at least 1000 μg/m²/day; at least 1080 μg/m²/day; at least 1430 μg/m²/day; at least 1905 μg/m²/day; or at least 2000 μg/m²/day. These values can form the start and end points for dosage ranges, for example 200-2000 μg/m²/day.

Preferred ranges for dosages may include 40-2000 μg/m²/day; 80-1000 μg/m²/day; 135-1000 μg/m²/day; 200-1000 μg/m²/day; or 470-870 μg/m²/day. Still more preferred doses may include one or more of: 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000, 1010, 1020, 1030, 1040, 1050, 1060, 1070, 1080, 1090, 1100, 1110, 1120, 1130, 1140, 1150, 1160, 1170, 1180, 1190, 1200, 1210, 1220, 1230, 1240, 1250, 1260, 1270, 1280, 1290, 1300, 1310, 1320, 1330, 1340, 1350, 1360, 1370, 1380, 1390, 1400, 1410, 1420, 1430, 1440, 1450, 1460, 1470, 1480, 1490, 1500, 1510, 1520, 1530, 1540, 1550, 1560, 1570, 1580, 1590, 1600, 1610, 1620, 1630, 1640, 1650, 1660, 1670, 1680, 1690, 1700, 1710, 1720, 1730, 1740, 1750, 1760, 1770, 1780, 1790, 1800, 1810, 1820, 1830, 1840, 1850, 1860, 1870, 1880, 1890, 1900, 1910, 1920, 1930, 1940, 1950, 1960, 1970, 1980, 1990 or 2000 μg/m²/day or a dosage range in which one of these values forms the start point and another value forms the end point of the dosage range, for example one or more of 40-50 μg/m²/day; 50-6 μg/m²/day; 60-70 μg/m²/day; 70-8 μg/m²/day; 80-90 μg/m²/day; 90-100 μg/m²/day; 100-110 μg/m²/day; 110-120 μg/m²/day; 120-130 μg/m²/day; 130-140 μg/m²/day; 140-150 μg/m²/day; 150-160 μg/m²/day; 160-170 μg/m²/day; 170-180 μg/m²/day 180-190 μg/m²/day; 190-200 μg/m²/day; 200-210 μg/m²/day; 210-220 μg/m²/day; 220-230 μg/m²/day; 230-240 μg/m²/day; 240-250 μg/m²/day; 250-260 μg/m²/day; 260-270 μg/m²/day; 270-280 μg/m²/day; 280-290 μg/m²/day; 290-300 μg/m²/day; 300-310 μg/m²/day; 310-320 μg/m²/day; 320-330 μg/m²/day; 330-340 μg/m²/day; 340-350 μg/m²/day; 350-360 μg/m²/day.

A predetermined time interval may be provided between dosages. This may be provided in order to ensure that, on average, a desired concentration of RH1, or other compound described herein, is maintained in the patient's blood. Preferred time intervals may be any one or more of: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350 or 360 minutes. Alternatively, preferred time intervals may be any one or more of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167 or 168 hours.

It is possible that the time interval between dosages can be varied. For example, a dosing schedule may be provided in which a selected dose is administered daily (i.e. at 24 hour intervals) for a number of days (e.g. any of 1, 2, 3, 4, 5, 6 or 7 days) and then a further time interval (e.g. 1, 2, 3, 4, 5, 6 or 7 days) is provided during which no drug is administered (i.e. a ‘drug holiday’). Each period of drug administrations followed by ‘drug holiday’ may comprise one cycle of treatment. A dosing routine may be provided having any number of cycles, as desired to achieve treatment. For example, 1, 2, 3, 4, 5, 6, 7, 8, 9 or 10 cycles may be provided. The patient may then be removed from treatment, which of course, may be re-commenced if further treatment is considered necessary.

Treatment of any cancerous condition, including all cancer types, may be provided. In some aspects, treatment of cancerous conditions in which DT-Diaphorase (DTD) levels are upregulated and/or in which DTD is over-expressed may be provided. In some aspects, tumours to be treated may be solid tumours.

The cancerous condition may be any unwanted cell proliferation (or any disease manifesting itself by unwanted cell proliferation), neoplasm or tumour or increased risk of or predisposition to the unwanted cell proliferation, neoplasm or tumour. The cancerous condition may be a cancer and may be a benign or malignant cancer and may be primary or secondary (metastatic). A neoplasm or tumour may be any abnormal growth or proliferation of cells and may be located in any tissue. Examples of tissues include the colon, pancreas, lung, uterus, stomach, kidney, testis, skin, blood or lymph.

Examples of such cancer types include lung, colon, NSCLC, stomach, colorectal, pancreatic endometrial, head, neck, breast, leukaemia, melanoma, renal cell, kidney, ovarian, prostrate, testicular, rectal, throat, tongue, gastric and intestinal cancer. In one preferred arrangement the cancerous condition is a lung neoplasm or tumour being a form of, or involved in the development of, a lung cancer, which may be NSCLC.

Cancerous conditions selected for treatment may be those that have proven to be resistant to (are refractory to) treatment with conventional chemotherapy or radiotherapy. Cancerous conditions may be those for which no conventional treatment exists.

Methods of treating patients, including human patients, having a cancerous condition are provided. Treatment may be by administration (e.g. by injection, orally, etc) of compounds or compositions according to the present invention, preferably of 2,5-diaziridinyl-3-hydroxymethyl-6-methyl-1,4-benzoquinone (RH1) or compositions containing 2,5-diaziridinyl-3-hydroxymethyl-6-methyl-1,4-benzoquinone (RH1). The differential in DTD expression between neoplastic and normal tissue preferably allows drug activation at the site of the tumour and minimises normal tissue toxicity.

The elevated levels of DTD in metastatic tissue makes these tissues a good target for treatment. Accordingly, some aspects of the invention include treatment of metastatic tissue.

The patient to be treated may be any animal or human. The patient may be a non-human mammal, but is more preferably a human patient. The patient may be male or female.

First Medical Use

In a related aspect, the present invention provides RH1 or a compound of formula I, or a pharmaceutically acceptable salt thereof, for use in a method of medical treatment of the human or animal body.

Preferably the method of medical treatment is treatment of a cancerous condition.

In a related aspect, the present invention provides RH1 or a compound of formula I, or a pharmaceutically acceptable salt thereof, for use in a method of medical treatment of the human or animal body wherein the patient has a condition which is known to exhibit over-expression of DTD.

Second Medical Use

In a further related aspect, the present invention provides the use of RH1 or a compound of formula I or a pharmaceutically acceptable salt thereof in the manufacture of a medicament for the treatment of a cancerous condition.

Another aspect of the present invention pertains to use of RH1 or a compound of formula I or a pharmaceutically acceptable salt thereof in the manufacture of a medicament, for the treatment of cancerous conditions which are characterised by cellular over-expression of DTD, and/or increased DTD activity, in cancerous cells relative to non-cancerous cells, as discussed herein.

Another aspect of the present invention pertains to a kit comprising (a) the compound, preferably provided as a pharmaceutical composition and optionally in a suitable container and/or with suitable packaging; and (b) instructions for use, for example, written instructions which may describe how to administer the compound/composition and/or the dosage to be administered and the time interval between dosages. Suitable dosage amounts and time intervals between dosages are described herein.

The invention includes the combination of the aspects and preferred features described except where such a combination is clearly impermissible or expressly avoided.

Aspects and embodiments of the present invention will now be illustrated, by way of example, with reference to the accompanying figures. Further aspects and embodiments will be apparent to those skilled in the art. All documents mentioned in this text are incorporated herein by reference.

### BRIEF DESCRIPTION OF THE FIGURES

FIG. 1 shows a graph of RH1 drug dosimetry results in human lymphocytes. The graph shows percentage (%) DNA crosslinking, as measured by the Comet-X assay, against RH1 concentration (nM). Optimal DNA cross-linking is shown in the range 50 to 100 nM RH1.

FIG. 2 shows, by way of example, the results of a Comet-X assay.

FIG. 3 shows a graph of % DNA cross-linking, as measured by the Comet-X assay, in peripheral blood lymphocytes (PBL) in five patients on days 1 and 5 of testing. T1=pre-dose, T2=5 mins post dose, T3=10 mins post dose, T4=20 mins post dose, T5=40 mins post dose, T6=1 hour post dose, T7=2 hours post dose, T8=4 hours post dose, T9=8 hours post dose, T10=10 hours post dose.

FIG. 4 shows pharmacokinetic data for RH1 in five patients receiving doses of RH1 of 40, 80, 135, 200 and 265 μg/SqM respectively. Patient RH1 plasma concentration (pg/μl) against time (minutes) is shown.

FIG. 5 shows the results of gel electrophoresis as performed as part of an RFLP assay.

FIG. 6 illustrates schematically a DCPIP assay.

FIG. 7 (A) shows representative images of PBL QC standards subjected to the Comet-X assay. (I) Control, (II) Irradiated, low dose RH1 (10 nM), (III) Irradiated+low dose RH1 (50 nM), (IV) Irradiated+high dose RH1. Cross-linking by RH1 reduces the extent of the radiation induced comet “tail”; and (B) DNA cross-linking observed in patients 1-12 at days 1 and 5 following RH-1 treatment.

FIG. 8 (A) NQO1 genotyping of patients 1-12. QC samples are representative genotypes. (B) Table of NQO1 genotyping of patients 1-12.

FIG. 9 Pharmacokinetic and pharmacodynamic data for RH1 in patients 1-12. P1D1=patient 1 day 1.

FIG. 10 Pharmacokinetic and pharmacodynamic data for RH1 in patients 1-12. (A) patients 1-4, (B) patients 5-8, (C) patients 9-12. P1D1=patient 1 day 1.

### DETAILED DESCRIPTION OF THE INVENTION

Specific details of the best mode contemplated by the inventors for carrying out the invention are set forth below, by way of example. It will be apparent to one skilled in the art that the present invention may be practiced without limitation to these specific details.

As discussed above, a number of quinones have been suggested for the treatment of cancer and some of these have been tested by the present inventors to assess their suitability as cross-linking agents and to assess toxicity effects. The results of these tests are set out in Table 2.

TABLE 2

| | | DTD | Reason for |
|---|---|---|---|
| Name | Compound | substrate | Failure |
| Mitomycin C | Benzoquinone | Poor | N/A |
| E09 | Indolquinone | Moderate | Renal |
| 3-HYDROXYMETHYL- | | | toxicity |
| 5-AZIRIDINYL- | | | |
| 1METHYL-2-[1H- | | | |
| INDOLE-4,7- | | | |
| DIONE]-PROPANOL | | | |
| AZQ | Aziridinylbenzo- | Moderate | No clinical |
| 3,6-diaziridinyl- | quinone | | benefit |
| 2,5-bis- | | | |
| (carboethoxyamino)1,4- | | | |
| benzoquinone | | | |
| BZQ | Aziridinylbenzo- | Poor | No clinical |
| 3,6-diaziridinyl- | quinone | | benefit |
| 2,5-bis-2(- | | | |
| hydroxyethylamino)1,4- | | | |
| benzoquinone | | | |
| MeDZQ | Aziridinylbenzo- | Moderate | Poor |
| ‘Me’-3,6- | quinone | | solubility |
| diaziridinyl-1,4- | | | |
| benzoquinone | | | |

The first bioreductive drug studied was mitomycin C which is traditionally used in chemotherapy for NSCLC. It was found that Xenografts derived from NSCLC cell lines with high levels of DTD were more susceptible to the cytotoxic effects of the antitumour quinone mitomycin C than those derived from SCLC cell lines with low levels of DTD. However, it is a relatively poor substrate for DTD and is pH-dependent. The dose limiting toxicity (DLT) was found to be myelosuppression.

RH-1 was found to be a good substrate for DTD and it is water soluble. Indeed, it has a water solubility 225 times that of mitomycin C.

### Phase I Clinical Trial of RH-1 (2,5-diaziridinyl-3-hydroxymethyl-6-methyl-1,4-benzoquinone)

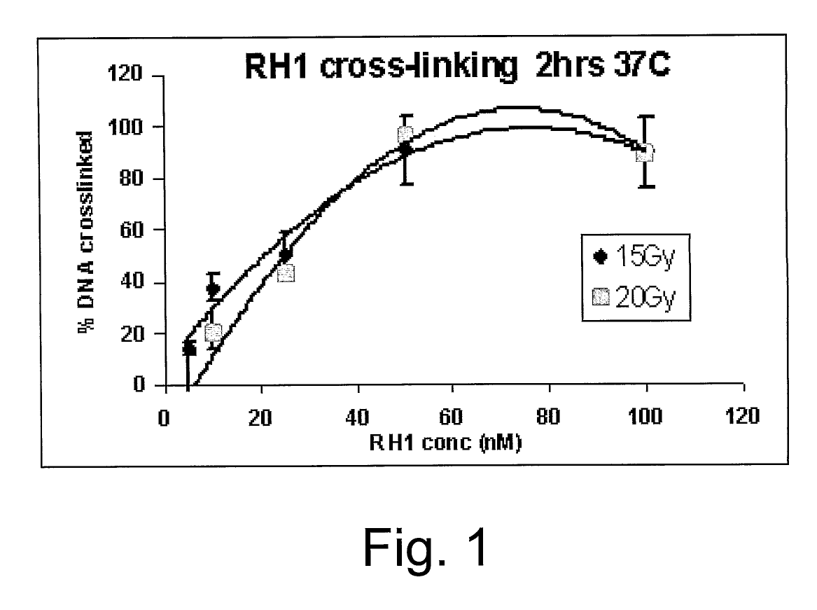

RH-1 is activated by NAD(P)H: Quinone acceptor oxidoreductase (NQO1; DT-diaphorase, DTD). DTD is often over-expressed in lung, colon, liver and breast tumours. RH-1 is undergoing Phase I trial at the Christie Hospital, UK. The pharmacokinetic (PK) requirements of this trial required assays that detect nM levels of RH-1 in serum using LC-MS. The extraction and quantitation of RH-1 from human plasma was validated and a limit of detection of 1 ng/ml reached. RH-1 PK were calculated and a linear relationship established between drug dose and area under the curve. Clearance values did not appear to be saturable even at the highest drug dose studied. FIG. 1 shows the results of the drug dosimetry study. The plasma half-life of RH-1 is between 2-12 min in the patient samples analysed so far.

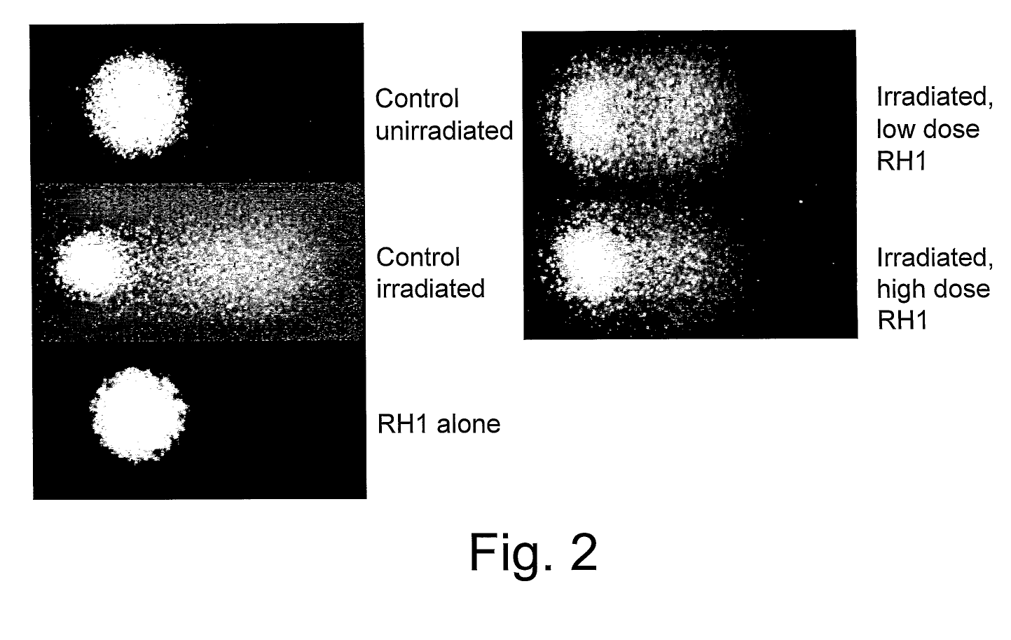

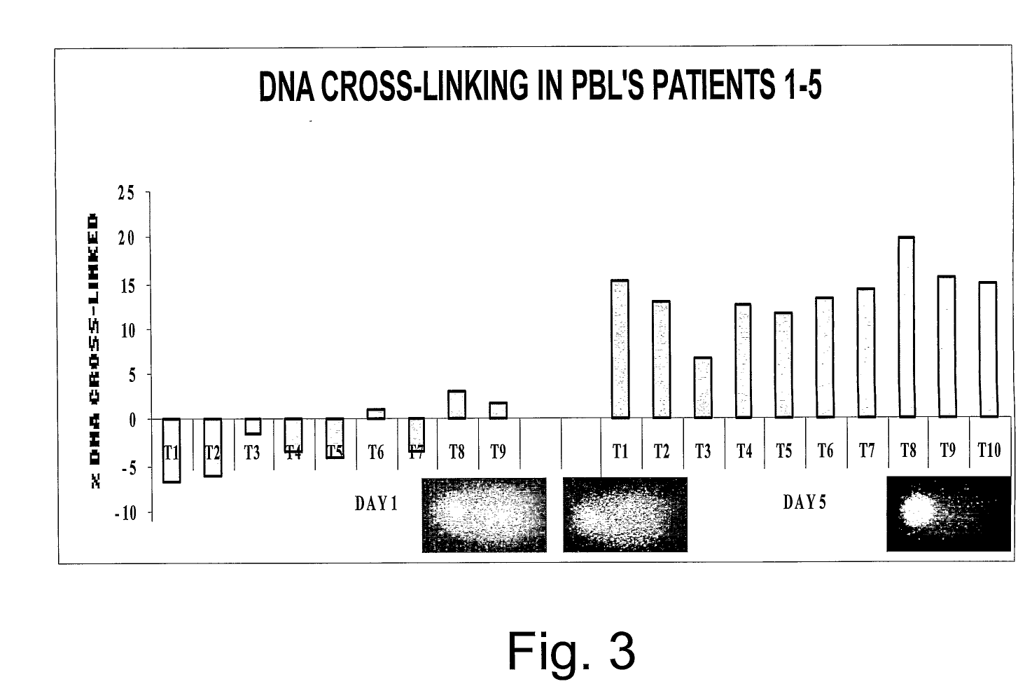

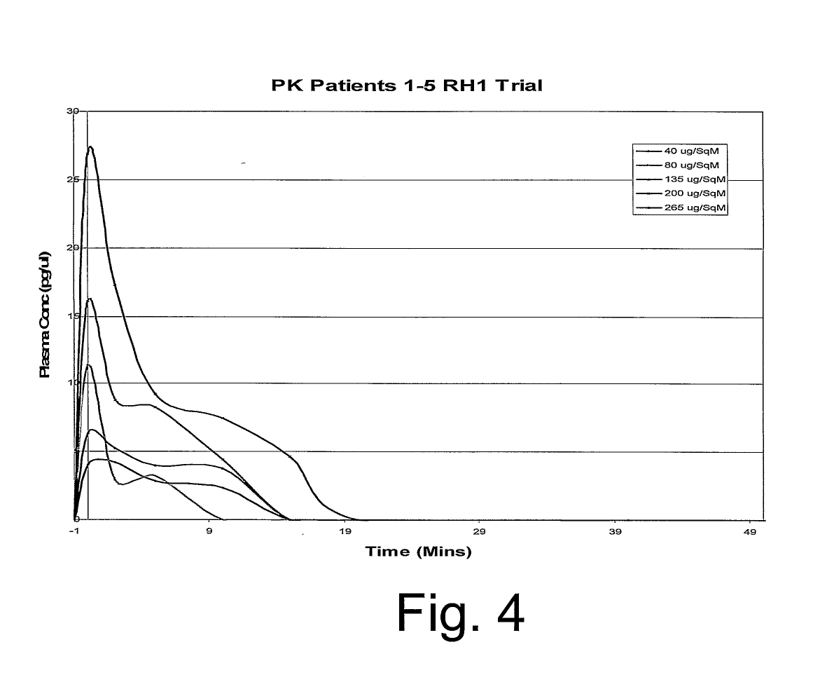

A modified version of the single cell gel electrophoresis comet assay, the Comet-X, specifically detects DNA cross-linking in individual cells and has been validated for clinical trial use. 9 patients have been treated with RH-1 and PMBCs (peripheral blood lymphocytes) isolated from pre-infusion and post infusion time points on both day 1 and day 5 of treatment were subjected to Comet-X assay (FIG. 2). From the data obtained, accumulative DNA damage appears to be occurring in PBMC's over the 5 day infusion period leading to significant DNA cross linking (30%) by day 5 (FIG. 3). Other PD (pharmacodynamic) assays used in the trial include assessment of DTD levels and activity and RFLP genotyping of the NQO1 gene.

The RH1 trial involved administering RH1 to patients having histologically proven solid tumours that were refractory to conventional treatment or for whom no conventional treatment exists. The WHO performance status of these patients was 0 or 1. WHO performance status is an indicator of a patients overall level of well being/activity. A WHO performance status of 0 means that the patient is fully active, able to carry out all normal activity, without restriction. A WHO performance status of 1 means the patient is restricted in physically strenuous activity but ambulatory and able to carry out work of a light or sedentary nature, e.g. light house work, office work.

RH1 was administered intravenously on a daily schedule for 5 day periods following a dose escalation scheme. Treatment was repeated on a 21 day cycle. A dose escalation scheme was used to try to establish dose-limiting toxicity. Predicted toxicity includes myelosuppression, emesis, renal toxicity and local injection site irritation. Safety and tolerability were monitored each cycle. Anti-tumour response was assessed after each 2 cycles using RECIST criteria.

Phase I clinical trials required validated assays for PK by mass spec, DNA cross-linking by Comet-X, patient genotype by Restriction Fragment Length Polymorphism (RFLP) and DTD levels (by DCPIP). Subsequent assays are to be validated for IHC, WB and PCR.

Twelve patients have been enrolled on this trial. Tumour types include: NSCLC, colorectal carcinoma and gastric carcinoma. Patient data is set out below in Table 3.

TABLE 3

| | | | | | Total |
|---|---|---|---|---|---|
| | | | Primary | | no. of |
| Patient | | | Diagnosis and | Dose | cycles |
| No. | Sex | Age | stage | (μg/m²/d) | given |
| 001 | M | 65 | Lung Cancer | 40 | 2 |
| | | | (NSCLC) stage IV | | |
| 002 | M | 58 | Colorectal | 80 | 2 |
| | | | cancer | | |
| | | | (adenocarcinoma- | | |
| | | | colon) stage IV | | |
| 003 | M | 65 | Gastric cancer | 135 | 2 |
| | | | (adenocarcinoma- | | |
| | | | stomach) stage IV | | |
| 004 | M | 58 | Colorectal | 200 | 2 |
| | | | cancer (colon | | |
| | | | carcinoma) stage IV | | |
| 005 | M | 69 | Colorectal stage IV | 265 | 6 |
| 006 | M | 55 | Stomach cancer | 350 | 4 |
| | | | stage IV | | |
| 007 | M | 64 | Colon carcinoma | 460 | 2 |
| | | | stage IV | | |
| 008 | M | 72 | Pancreatic | 610 | 2 |
| | | | carcinoma stage IV | | |
| 009 | M | 65 | Melanoma stage IV | 810 | 2 |
| 010 | M | 30 | Melanoma stage IV | 1080 | 4 |
| 011 | M | 69 | Adenocarcinoma- | 1430 | 2 |
| | | | colon stage IV | | |
| 012 | F | 40 | Renal cell | 1905 | 3 |
| | | | carcinoma stage IV | | |
| 013 | F | 68 | Colon carcinoma | 1905 | 2 |
| | | | stage IV | | |
| 014 | M | 60 | Adenocarcinoma | 1905 | 1 |
| | | | colon stage N/K | | |

The first 4 patients had no treatment-related toxicity. Patient 5 had grade I thrombocytopenia and grade I renal impairment. Pharmacokinetic analysis of plasma samples taken on day 1 and day 5 of cycle 1 shows detectable levels of drug with a half-life of approximately 6 minutes. Patients lymphocytes exposed to RH1 on infusion were analysed using the comet-X assay, which detects DNA interstrand cross-links. FIG. 3 shows the results.

Statistical analysis of comet-X assays from all patients on day 5 shows significantly more cross-linking than on day 1, as illustrated in FIG. 3. Pharmacokinetic analysis of plasma samples taken on day 1 and day 5 of cycle 1 show detectable levels of drug with a half-life of approximately 6 minutes for clearance from the blood. The peak levels range from 17 to 113 nM with escalating dose. These dose levels are consistent with those causing significant biological activity in vitro.

Comet Assay

This is a single cell gel electrophoresis assay that was first described in 1984 as a microelectrophoretic technique for the direct visualisation and quantification of DNA damage in individual cells (Ostling & Johanson, 1984).

The original technique only allowed detection of DNA strand breaks (dsbs) and so it has been modified to measure DNA cross-linking (“Retardation of radiation-induced DNA migration used as a surrogate measurement of cross-linking” Ward et al. Biochem. Pharmacol. 5, 459-64, 1997) and is known as the “comet-X assay”.

Examples of Comet-X data are given in FIG. 2. The formation of DNA cross-links causes retardation of the DNA tail. With higher doses of RH1 there is a greater retardation.

The Comet assay is a sensitive method of detecting DNA breaks in single cells. The cells are embedded in agarose and spread onto microscope slides. After lysis and de-proteinisation, the cells are subjected to alkaline unwinding at high pH (12.5). The DNA becomes relaxed and unwinds at points where DNA single and double strand breaks occur. The cells are then finally subjected to micro-electrophoresis during which relaxed and damaged DNA migrates away from the nucleus resulting in the classic comet shape when visualised under microscopy. This migration of DNA is retarded when the DNA has been subjected to interstrand cross-linking by drugs such as RH1. In the comet-X assay used in this trial, control non-drug treated lymphocytes from the patient's pre-infusion are subjected to gamma radiation to introduce a fixed number of DNA breaks into each cell. This treatment results in a fixed amount of migration under electrophoresis quantified as the percentage (%) of DNA in the tail of the comet image. Patients lymphocytes exposed to RH1 on infusion are collected and irradiated with the same dose of gamma radiation as the control pre-infusion lymphocytes. It was expected that interstrand cross-links produced by RH1 will retard the migration of DNA during electrophoresis resulting in less DNA in the tail of the comet compared to the irradiation only controls. Results are expressed as % DNA cross linked.

To date 12 patients have been treated with RH1 and peripheral blood lymphocytes isolated from pre-infusion and post infusion time points on both day 1 and day 5 of treatment. These lymphocytes have been subjected to the Comet-X assay described above and the amount of DNA present in the tail of the comets following irradiation has been measured. Internal QC samples have been run each time a patients samples have been processed. The data has been pooled into exposure times (short 5-10 minutes, medium 40-120 minutes and long 4-24 hrs exposure) and dose cohorts, (1) 40-135 μg/m², (2) 200-326 μg/m², (3) 410-810 μg/m², and (4) 1080-1905 mg/m² ranges for ease of analysis.

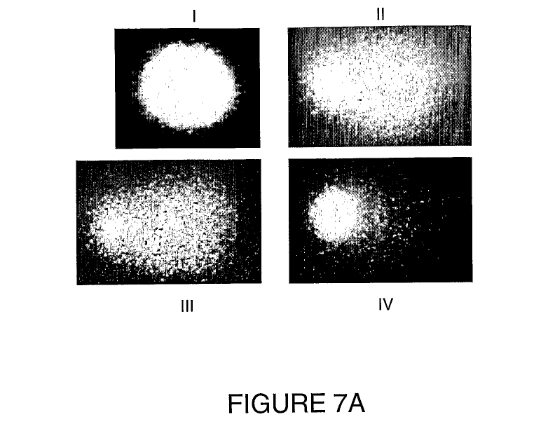

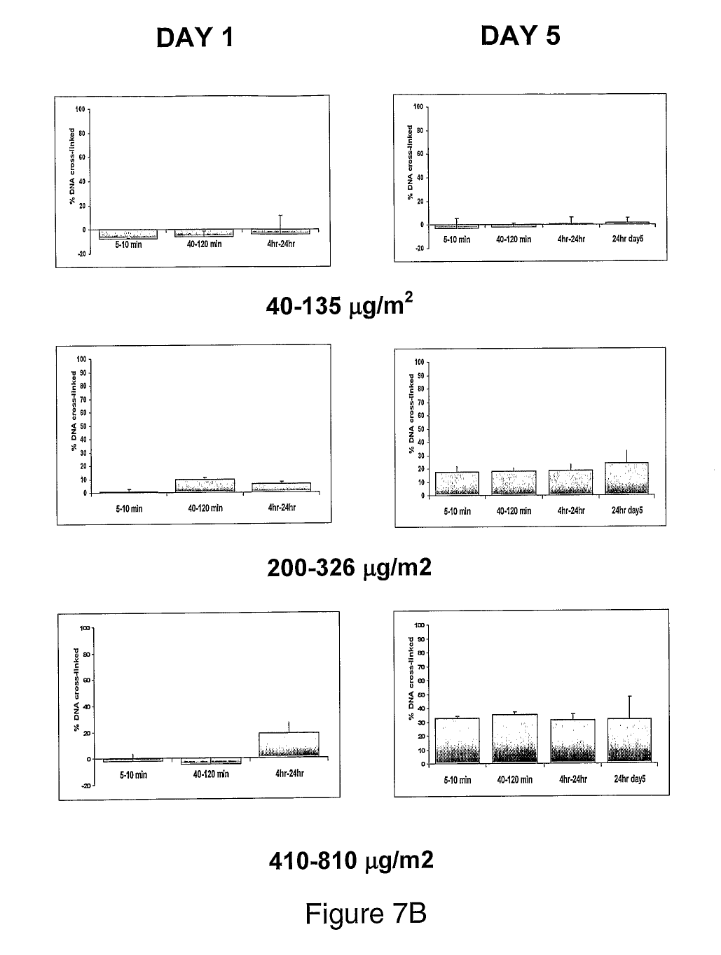

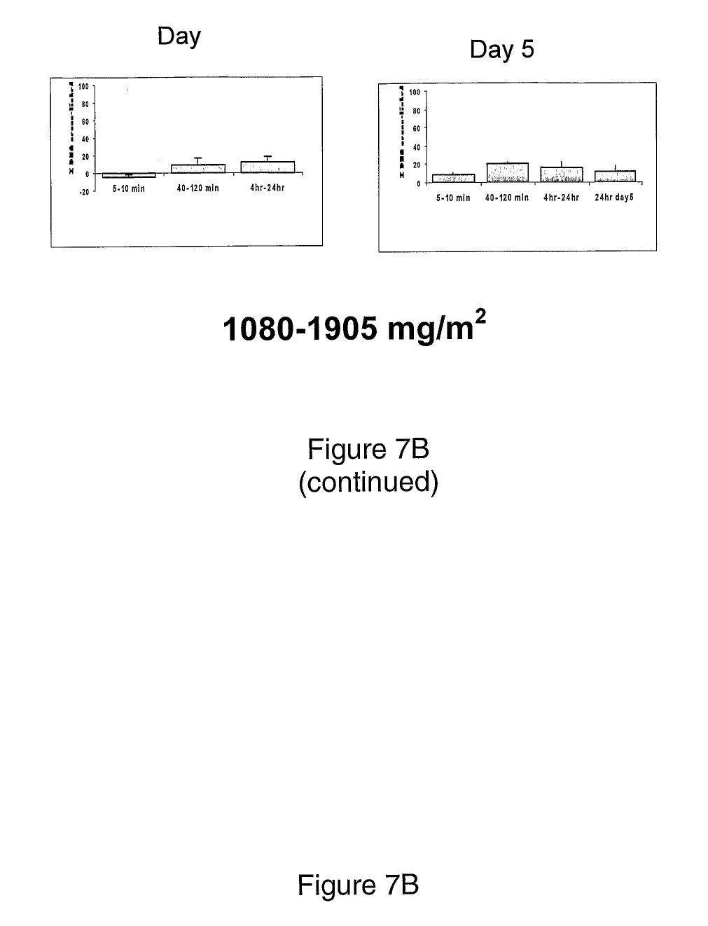

The pooled data for all patients show no DNA cross-linking in the low dose cohort either day 1 or day 5 of treatment (FIG. 7B). Indeed when the % DNA cross-linked is calculated a negative value is arrived at suggesting that additional strand breaks are being produced by the drug, possibly by redox cycling or oxidative stress.

Day 5 results, however, show evidence of DNA cross-linking at all time points particularly in the later (8 hour, 24 hour) samples (FIG. 3 and FIG. 7B). In patients the distribution peaks show 70-80% DNA in the tail similar in distribution to the irradiated control. However, by day 5 the PBLC population shows peaks at 60-70% DNA in the tail suggesting low level cross-linking similar to the low dose internal controls. Statistical analysis of comets from all patients on day 5 show a significant difference to those measured on day 1 (p=0.002, T-test).

The medium dose cohort shows evidence of DNA cross-linking on day 1, particularly in the later (4 hr, 24 hr) samples, and throughout day 5. In terms of length of exposure Student T-test analysis shows no significant differences between the first and second dose groups on day 1 versus day 5, however the longer 4-24 hr exposure time points score just below significance (p=0.06). In contrast analysis of dose cohort's shows significant differences (p<0.05) between day 1 and day 5 samples when the first and second dose cohorts are compared, but no difference is seen between the higher dose cohorts.

It would appear that DNA damage in the form of strand breaks occurs at all time points on initial (day 1) treatment in the low dose cohort. This strand breakage is inferred from the negative cross linking values obtained. The comet-x assay incorporates an irradiation step to introduce a fixed number of strand breaks into the DNA. Consequently in the absence of significant strong interstrand cross linking additional strand breaks would be additive to the irradiation step. The cause of these breaks is not clear and may be the result of reactions either directly related to drug action i.e. redox cycling, or from general stress response pathways activated by treatment. This effect reduces significantly as the dose of RH1 increases and is absent entirely from medium and high dose cohorts by day 5. From the data obtained so far accumulative DNA damage appears to be occurring in PBMC's over the 5 day infusion period leading to significant DNA cross linking (30%) by day 5. However in the highest cohort (1080-1095 mg/m²) the degree of cross-linking drops to 15-20%. It is possible in this group of patients that high dose RH1 has depleted the most affected population of PBMC's leaving the moderately damaged cells intact. Indeed the isolated PBMC count for two out of the three patients in these cohorts was lower than had been previously observed. It is also possible that repair had taken place however, analysis of samples 24 hr post day 5 and on days 8, 15 and 21 show little evidence for significant repair. The comet-X assay has been able to demonstrate efficacy on skin biopsy lesions in the highest cohort treated.

The comet data from patients in the trial was correlated with PK parameters, other PD results, toxicity and response data.

Polled PBLs from the previous experiment were treated at 5, 10, 25, 50 and 100 nM RH1 for 2 hours at 37° C. and there was a non-drug treated sample too. The samples were irradiated at 15 and 20 Gy.

FIG. 1 shows the correlation of percentage DNA cross-linking with measured concentration of RH1. The dose response curves showed an increase in DNA cross-linking as the concentration of RH1 was increased to 50 nM.

FIG. 7B shows percentage DNA crosslinking over time for a range of doses of RH1.

There is increasing interest in the use of this assay as a pharmacodynamic endpoint in clinical trials.

The assay was used to look for DNA cross-linking in PBLs and in tumour, and to correlate the findings with toxicity and response results.

Restriction Fragment Length Polymorphism (RFLP) Assay

NQO1 polymorphism has been identified in BE cells that have no functional DTD (Traver et al. Cancer Res. 52, 797-802, 1992). A Single Nucleotide Polymorphism (SNP), which is believed to cause the polymorphism comprises a homologous base substitution (C to T) at position 609 on the NQO1 gene results in a proline to serine substitution and thus deletion of exon 4. Exon 4 codes for the quinone substrate and thus this means that active DTD is not expressed. The active DTD expressed is much less stable (there is a change in the enzyme conformation which leads to reduced FAD binding affinity) and is broken down by the UPP in 1.2 hours (c.f. 18 hours for the normal enzyme).

Deletion mutagenesis in the NQO1 gene promoter identified several cis-elements, including antioxidant response element (ARE), xenobiotic response element and AP2 element, which regulate the expression and induction of NQO1.

The SNP exists in 4% of Caucasians and 20% of Asians (Kesley et al. Br. J. Cancer 76, 852-4, 1997). The incidence of SNP is increased in certain cancers. Patients having the SNP are susceptible to benzene and quinone toxicity.

Patients with the polymorphism were not excluded from the trial but one needs to know their status when analysing toxicity and efficacy.

Nevertheless, treatment of patients having the SNP may still be possible because the compound is also activated by 1e reductases. Furthermore, heterozygotes have intermediate activity and treatment may therefore be possible.

An RFLP assay was used to detect the polymorphism. The assay involved isolating DNA from whole blood, quantifying it with a Genespec microspectrophotometer (1.6-2.1) and amplifying by PCR. The amplified DNA was digested at HinF1 sites and gel electrophoresis conducted with ethidium bromide. The HinF1 site was created by point mutation.

For each of 10 NSCLC patients, PCR and DNA digest was repeated four times.

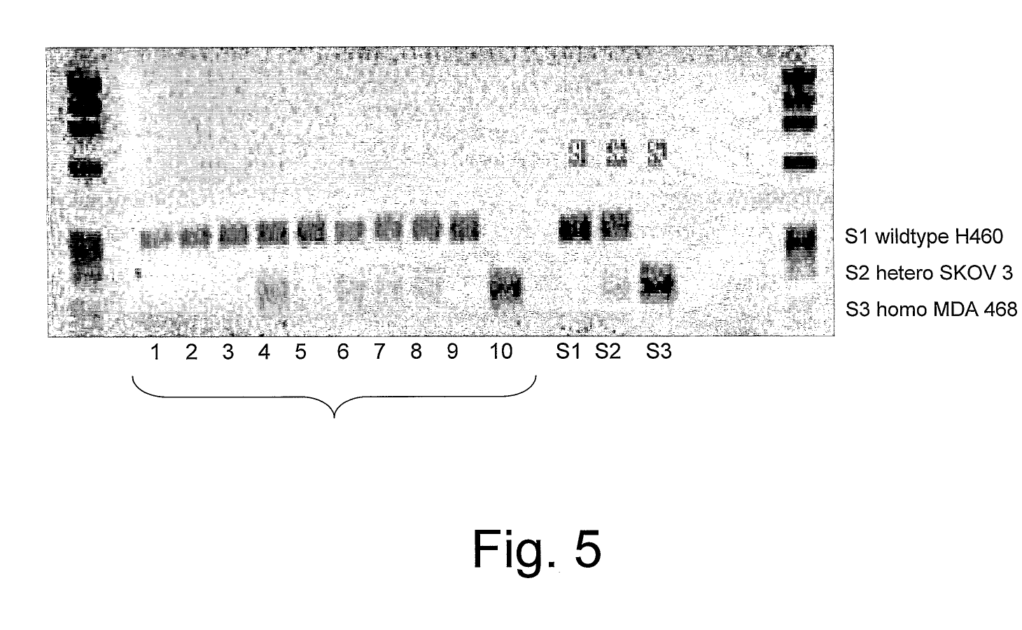

The internal controls were H460 (wildtype), SKOV3 (hetero) and MDA-468 (homo). Forward and reverse primers were mapped using sequencer software. FIG. 5 shows the gels for 10 nsclc patient samples and the three internal standards, S1 (wildtype), S2 (hetero) and S3 (homo). Samples 1, 2, 3, 5 and 9 were matched as wildtype, samples 4, 6, 7 and 8 as hetero and sample 10 as homo. These sequences were confirmed with DNA sequencing. The nucleotide sequence for the Human NAD(p)H:quinine oxidoreductase gene is available from the NCBI database (http://www.ncbi.nlm.nih.gov/) under accession number AH005427 (M81596.1 GI:808928).

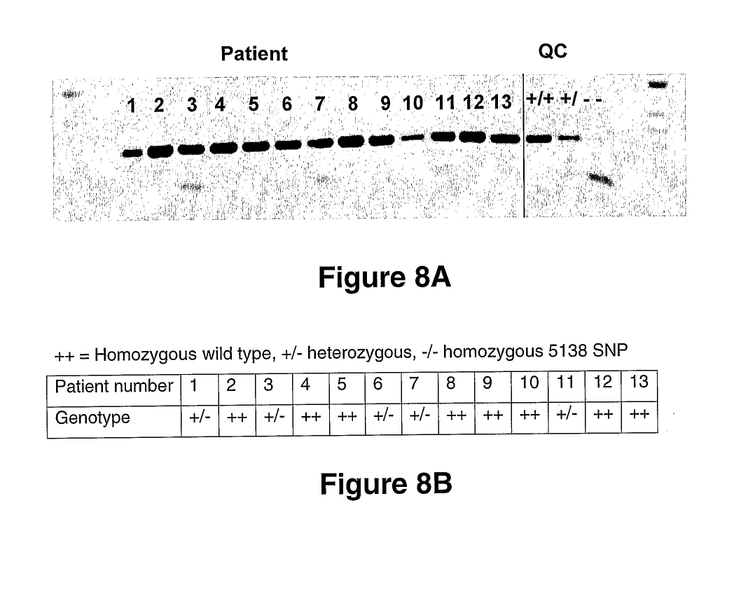

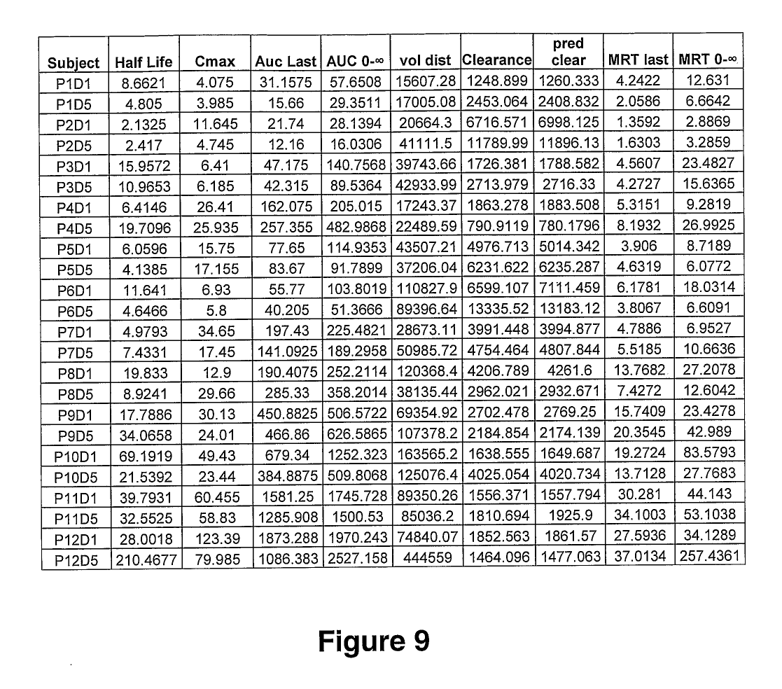

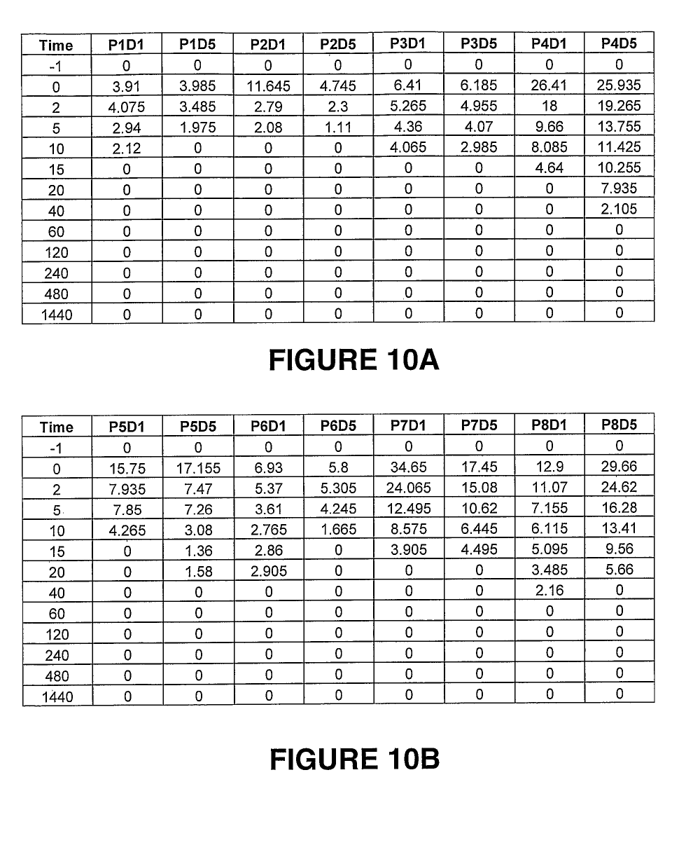

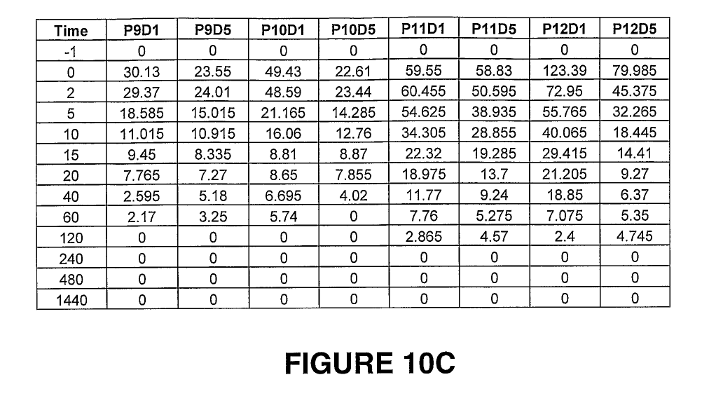

For 12 patients DNA extracted from samples was analysed using the validate RFLP assay. The assay results and genotyping are shown in FIGS. 8A and 8B. Five samples were found to be heterozygous for the C-T transition (SNP) at position 5138 in the genomic sequence (Jaiswal A K. Human NAD(P)H:quinone oxidoreductase (NQO1) gene structure and induction by dioxin. Biochemistry. 1991 Nov. 5; 30(44):10647-53, gene bank accession AH005427) whilst eight were found to be homozygous wild type. No samples were found to be positive for the 5138 SNP.

### 2,6-dichlorophenolindophenol (DCPIP) Assay

The DCPIP assay was used to assess levels of functional DTD. DCPIP is blue in colour and is reduced by DTD via the co-factor NADPH to a colourless solution. The rate at which the colour is lost is proportional to the activity of the DTD. DTD is specifically inhibited by the addition of dicumarol so if the assay is carried out in the presence of dicumarol, any remaining activity is due to one electron reductases. DTD activity is calculated by subtraction of one-electron activity from total activity.

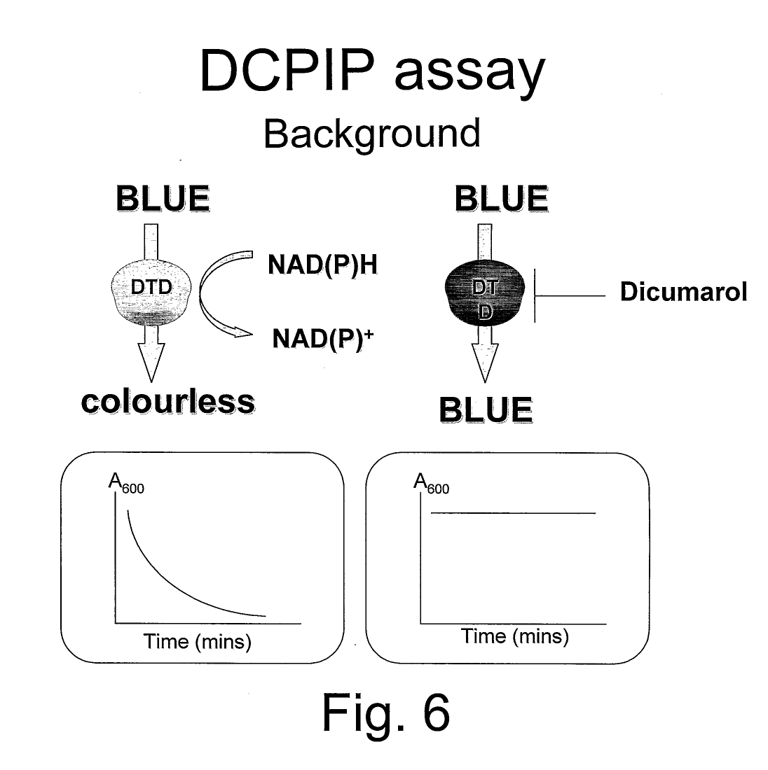

FIG. 6 illustrates the DCPIP assay schematically.

The results of measurements of functional DTD levels in tumour specimens was correlated with Western blotting, immunohistochemistry (IHC) and reverse transcriptase PCR (RT-PCR).

Ongoing Work

14 patients have now been recruited into the study.

Patient 007 has been enrolled at dose level 7. So far there has been evidence of toxicity. The results indicate that stabilisation of the disease may be possible. The pharmacokinetic data is consistent. Results so far suggest that increasing DNA cross-linking occurs with increasing doses of RH1. Genotype and biopsy data is awaited.

Chemical Synthesis of RH1

RH1 (2,5-diaziridinyl-3-hydroxymethyl-6-methyl-1,4-benzoquinone) may be synthesised as follows.

To a stirred solution of 2-hydroxymethyl-5-methyl-1,4-benzoquinone (10 g, 65.8 mmol) in ethanol (250 ml), under N₂ at 0° C., was added aziridine (6.8 ml, 5.66 g, 131.6 mmol). After 20 mins the solution was allowed to rise room temperature and stirred for a further 5 hours. The solvent was then reduced in vacuo to approximately 100 mls and then cooled on ice. The resulting precipitate was filtered and washed with ice cold ethanol (50 ml). A further crop could be obtained by reducing the solvent to about 50 ml, cooling and filtering again. The combined yield was 2.813 g of dark red crystals. (18.3%, m.p. 178-9.degree. C.); ¹H NMR (200 MHz, CDCl₃): δ4.56 (2H, d, J=6 Hz, CH₂), 2.64 (1H, t, J=6 Hz, OH), 2.38 (4H, s, Az), 2.28 (4H, s, Az), 2.0 (3H, s, CH₃); MS El m/z: 234 (M⁺), 219, 191, 177, 163, 149; Vₘₐₓ (KBr disc): 3483, 2995, 1637, 1585, 1383, 1300, 1159; HREIMS. Found 234.1005 C₁₂H₁₄N₂O₃ requires 234.1004.

RH1 is easily synthesized with very high purity (>99%). RH1 is readily soluble in aqueous solution (solubility in phosphate buffered saline is >0.5 mg/ml at 25° C.). The RH1 solutions are very stable with a half life of RH1 in phosphate buffer (0.1 M, pH=7) of more than 2 days at 25° C. The free hydroxyl group of RH1 accounts for its water solubility that leads to a shorter half-life in pharmacokinetics.

Benzoyl RH1

The benzoyl ester of RH1 (3,6-diaziridinyl-5-methyl-1,4-benzoquinone) may be synthesized as follows.

A solution of RH1 (50 mg, 0.21 mmol), benzoic acid (30 mg, 0.24 mmol), DCC (60 mg, 0.29 mmol) and DMAP (10 mg, 0.08 mmol) in DCM (10 ml) was stirred for 24 hrs. T.l.c. showed that all the RH1 had reacted and the solvent was removed in vacuo. The residue was then passed down a silica column using petroleum ether 40:60/ethyl acetate (3:1→42:1) as the eluent to yield a red solid. (51 mg, 71%, m.p. 149-50° C.); ¹H NMR (300 MHz, CDCl₃): δ8.02 (2H, m, Ar—H 2 and 6), 7.57 (1H, m, Ar—H 4), 7.43 (2H, m, Ar—H 3 and 5), 5.33 (2H, s, CH₂), 2.44 (4H, s, Az), 2.35 (4H, s, Az), 2.07 (3H, s, CH₃); MS (EI+) m/z: 338 (M⁺), 233, 218, 122, 105; Vₘₐₓ (film): 1716, 1643, 1587, 1384, 1300, 1269.

Acetyl RH1

Acetyl RH1 (2-Acetoxymethyl-3,6-diaziridinyl-5-methyl-1,4-benzoquinone) may be synthesized according to the following method.

To a stirred solution of RH1 (40 mg, 0.17 mmol) in pyridine (2 mls) was added acetic anhydride (200 μl, 216 mg, 2.1 mmol). After seven hours the reaction mixture was poured into water (20 mls) and extracted with ether. The combined organic fractions were dried (Na₂SO₄) and the solvent removed in vacuo. The resulting solid was passed down a silica column using chloroform:methanol (24:1) as the eluent to yield a red precipitate. (32 mg, 68%, m.p. 114-5° C.); ¹H NMR (400 MHz, CDCl₃): δ5.08 (2H, s, CH₂), 2.41 (4H, s, Az), 2.34 (4H, s, Az), 2.09 (3H, s, CH₃), 2.05 (3H, s, CH₃); MS (EI+) m/z: 276 (M⁺), 234, 217, 205, 149, 81; Vₘₐₓ (film): 1738, 1643, 1587, 1384, 1300, 1230.

1-7. (canceled)

8. A method of treating a cancerous condition in a patient in need of treatment thereof comprising the step of administering to the patient a therapeutically effective amount of 2,5-diaziridinyl-3-(hydroxymethyl)-6-methyl-1,4-benzoquinone (RH1).

9. The method of claim 8, wherein the compound is administered as a pharmaceutical composition comprising said compound.

10. The method of claim 9, wherein the pharmaceutical composition comprises a pharmaceutically acceptable carrier, adjuvant or diluent.

11. The method of claim 8, wherein the therapeutically effective amount is one that produces at time t₀, immediately following administration of RH1, a concentration of RH1 in the patient's blood which is in the range 1 to 200 nM.

12. The method of claim 8, wherein the therapeutically effective amount is one that produces at time t₀, immediately following administration of RH1, a concentration of RH1 in the patient's blood which is in the range 30 to 120 nM.

13. The method of claim 8, wherein the therapeutically effective amount is one that produces at time t₀, immediately following administration of RH1, a concentration of RH1 in the patient's blood which is in the range 50 to 90 nM.

14. The method of claim 8, wherein the therapeutically effective amount is one that produces at time t₀, immediately following administration of RH1, a concentration of RH1 in the patient's blood which is selected from one or more of: 40-45 nM; 45-50 nM; 50-55 nM; 55-60 nM; 60-65 nM; 65-70 nM; 70-75 nM; 75-80 nM; 80-85 nM; or 85-90 nM.

15. The method of claim 8, wherein the therapeutically effective amount is in the range 40 μg/m²/day to 350 μg/m²/day.

16. The method of claim 8, wherein the therapeutically effective amount is selected from one or more of: 40-50 μg/m²/day; 50-60 μg/m²/day; 60-70 μg/m²/day; 70-80 μg/m²/day; 80-90 μg/m²/day; 90-100 μg/m²/day; 100-110 μg/m²/day; 110-120 μg/m²/day; 120-130 μg/m²/day; 130-140 g/m²/day; 140-150 μg/m²/day; 150-160 μg/m²/day; 160-170 μg/m²/day; 170-180 μg/m²/day 180-190 μg/m²/day; 190-200 μg/m²/day; 200-210 μg/m²/day; 210-220 μg/m²/day; 220-230 μg/m²/day; 230-240 μg/m²/day; 240-250 μg/m²/day; 250-260 μg/m²/day; 260-270 μg/m²/day; 270-280 μg/m²/day; 280-290 μg/m²/day; 290-300 μg/m²/day; 300-310 μg/m²/day; 310-320 μg/m²/day; 320-330 μg/m²/day; 330-340 μg/m²/day; 340-350 μg/m²/day; 350-360 μg/m²/day.

17. A kit comprising:
(a) a pharmaceutical composition comprising 2,5-diaziridinyl-3-(hydroxymethyl)-6-methyl-1,4-benzoquinone (RH1); and
(b) instructions for use of the composition in the treatment of a cancerous condition, said instructions indicating a suitable dosage amount to be administered and optionally the time interval between dosages.

18. A kit according to claim 17 wherein the dosage amount is one that produces at time t₀, immediately following administration of RH1, a concentration of RH1 in the patient's blood which is in the range 1 to 200 nM.

19. A kit according to claim 17 wherein the dosage amount is one that produces at time t₀, immediately following administration of RH1, a concentration of RH1 in the patient's blood which is in the range 30 to 120 nM.

20. A kit according to claim 17 wherein the dosage amount is one in the range 40 μg/m²/day to 350 μg/m²/day.

21. The method according to claim 8 wherein said cancerous condition is one exhibiting DTD activity.

22. The method according to claim 8 wherein DTD activity and/or expression is upregulated in said cancerous condition.

23. The method according to claim 8 wherein the cancerous condition is a solid tumour.

24. The method or kit according to claim 8 wherein the cancerous condition is in a human patient.
